(19) United States
(12) Patent Application Publication
(10) Pub. No.: US 2017/0258540 A1
(43) Pub. Date: Sep. 14, 2017

(54) CONTROL SYSTEM FOR ELONGATE INSTRUMENT

(71) Applicant: Eric D. Blatt, McLean, VA (US)

(72) Inventor: Eric D. Blatt, McLean, VA (US)

(21) Appl. No.: 15/453,437

(22) Filed: Mar. 8, 2017

Related U.S. Application Data
(60) Provisional application No. 62/305,171, filed on Mar. 8, 2016.

(51) Int. Cl. A61B 34/00; A61B 17/00; A61M 25/01

(52) U.S. Cl. CPC A61B 34/71; A61B 34/76; A61M 25/0105; A61B 17/00234; A61M 2205/50

(57) ABSTRACT

A system for performing minimally invasive surgery includes a control tool and an elongate member for insertion into a body lumen. The control tool includes a first control tool bending segment and a first control tool transducer configured to generate a first control tool deflection signal based on manipulation of the first control tool bending segment. The elongate member includes a first elongate member bending segment at a distal portion and a first elongate member actuator configured to apply a force at the first elongate member bending segment. The system further includes a processor unit in communication with the control tool and the elongate member. Upon receipt of the deflection signal, the processor generates a first elongate member actuator signal configured to cause the first elongate member bending segment to move in accordance with the deflection signal.

### CROSS-REFERENCE TO RELATED APPLICATIONS

This application incorporates by reference and claims the benefit of priority to U.S. Provisional Application No. 62/305,171, filed on Mar. 8, 2016.

### BACKGROUND OF THE INVENTION

The present subject matter relates generally to methods and apparatuses for selectively manipulating an elongate instrument. More specifically, the present invention relates to methods and apparatuses for providing enhanced control of elongate medical tools including but not limited to guidewires, sheaths, catheters, endoscopes, laporoscopic tools, etc.

Minimally invasive surgical procedures have become increasingly common due to their potential to reduce complications and discomfort while accelerating recovery time. These procedures typically involve inserting an elongate device into an opening in the body. In the case of laporoscopic surgery, the physician creates an opening to directly access a tissue region, and then inserts one or more flexible or rigid instruments to the target tissue site and manipulates the instruments to perform the surgery. In intraluminal procedures, an elongate instrument is inserted into a preexisting body lumen, such as a blood vessel, esophagus, intestine, or urological, reproductive or other lumen. A flexible elongate instrument is then passed through the body lumen and advanced to the target site, where the instrument may then be manipulated to perform an interventional technique.

Such procedures frequently involve precisely manipulating the distal tip of the elongate instrument. For example, in order to access the target site, the tip of an elongate instrument may need to be positioned through a stenosed valve, or through narrow ostium at an acute or otherwise difficult angle. Manipulability challenges may be further exacerbated by variations in patient anatomy. For example, blood vessels or other body lumens may be tortuous which may reduce the predictability and responsiveness of an instrument that is advanced through the tortuous anatomy. Previously implanted devices may also pose challenges. For example, a previously installed stent or graft may obstruct the opening to a branch vessel. Even after the tool has reached the target site, the distal end of the tool may need to be precisely manipulated in order to complete the procedure. For example, a particular region of tissue may need to be engaged by a grasper, stapler, ablation probe, or other interventional tool, or an implant may need to be released in a particular orientation or at a particular region of a lumen.

Current tools generally allow for only a single degree of bending at the distal end and rely on rotation about the longitudinal axis of the tool to control the direction of this bending. This does not allow the physician to freely control the position of the distal tip of the tool, nor does it allow the physician to control the angle of attack with which the tool engages the tissue or ostium. Additionally, because manipulation of existing tools depends on axial rotation of the tool about its longitudinal axis, these tools must be manufactured to ensure torsional stability which tends to increase cost. Even with this investment, tortious lumens may render axial rotation difficult, and may reduce the responsiveness and predictability of the tool's operation.

Accordingly, there is a need for methods and apparatuses for providing enhanced control at the distal tip of an elongate medical instrument, as described herein.

### BRIEF SUMMARY OF THE INVENTION

In order to meet the needs described above and others, the present disclosure describes methods and apparatuses for providing enhanced control at the distal tip of an elongate medical instrument.

In one embodiment of the invention, an enhanced control system may include a control tool, a processor unit, and an elongate instrument. A control tool may include one or more or joints that allow a physician to apply bending inputs to the control tool, and may further include one or more sensors that detect and measure such physician inputs. Thus, when a physician applies bending inputs to the control tool, the control tool may produce a control signal that may indicate to a processor unit the shape or series of bends that the physician has selected.

A processor unit may be configured to receive inputs from one or more sensors arranged on a control tool. The processor unit may be further configured to produce an actuation signal selected to cause a portion of an elongate instrument to bend in a manner that mirrors, simulates, or otherwise correlates to bending inputs received at the control tool.

An elongate instrument may include one or more actuators that may be configured to produce a bending force in response to one or more selectively applied signals or other stimuli. Upon receiving a selectively applied actuation signal from a processor unit, the actuators in the elongate instrument may bend in a manner that mirrors, simulates, or otherwise correlates to bending inputs received at the control tool. In this manner, a portion of an elongate instrument, such as the distal tip thereof, may be configured to bend to adopt a shape or configuration selected by a physician at a control tool. The actuators may comprise electroactive polymer (EAP) actuators. In one embodiment, the actuators may comprise ionic polymer-metal composite (IPMC) actuators that may be stimulated by electronic signals passing along conductors embedded along the length of the elongate instrument.

Embodiments of the invention may also provide improved haptic feedback to enable the physician to detect tissue structures that the elongate instrument engages, thereby enhancing physician control and improving patient safety. For example, an elongate instrument may include sensors arranged at one or more joints to measure bending at the joints. In some embodiments, the sensors may comprise flex gauges or strain gauges. In some embodiments, the sensors may produce a detection signal that indicates the degree of bending observed at the joints of the elongate instrument. In this manner, the system may be configured to measure any contact force applied to the elongate instrument by comparing an observed bending measurement to an expected bending measurement determined on the basis of a selectively applied actuation signal.

A control tool may include one or more actuators configured to apply a bending force to one or more joints in a control tool. A processor unit may be configured to receive inputs from one or more sensors arranged on an elongate instrument and may, in response to the received inputs, generate a feedback signal configured to cause actuators arranged on the control tool to apply bending forces that mirror, simulate, or otherwise correlate to bending inputs received from the elongate instrument. In this manner, a contact force applied to an elongate instrument by an external structure may be simulated or reproduced in a portion of a control tool that the physician grasps or otherwise observes. In such an embodiments, a physician may feel or otherwise detect tissue structures that the elongate instrument engages.

In another exemplary embodiment, a method for selectively controlling a portion of an elgonate instrument is provided. A user may selectively apply a bending input at a control tool. The control tool may include one or more joints such that each joint may include one or more sensors to detect a bending input applied by the user. The control tool may output a bending signal to a processor unit. The processor unit may receive the bending signal, and produce an actuation signal that may configured to cause one or more actuators in an elgonate instrument to bend in a manner that mirrors, simulates, or otherwise correlates to bending inputs received at the control tool.

In another exemplary embodiment, a method for providing haptic feedback at a control tool is provided. For example, an elongate instrument may be advanced to engage a tissue structure, such that the tissue structure applies a contact force to the elongate instrument. The elongate instrument may include sensors arranged at one or more joints to produce detection signals that indicate the degree of bending observed at the joints. A processor unit may be configured to calculate the contact force by comparing an observed bending measurement to an expected measurement that may be determined on the basis of a selectively applied actuation signal. The processor unit may generate a feedback signal configured to cause actuators arranged on a control tool to apply bending forces that mirror, simulate, or otherwise correlate to bending inputs received from the elongate instrument. In this manner, a contact force applied to an elongate instrument by an external structure may be simulated or reproduced in a portion of a control tool that the physician grasps or otherwise observes.

An object of the invention is to provide a solution to allow the distal end of an elongate instrument to be manipulated quickly and precisely.

Another object of the invention is to provide a solution to offer a physician greater freedom of motion as he or she steers or manipulates the elongate instrument.

A further object of the invention is to provide a control tool that is capable of reproducing contact forces applied at a distal tip of an elongate instrument.

A further object of the invention is to provide an improved control apparatus and method that eliminates the need to axially torque an elongate instrument in order to position the instrument tip in a desired location.

An advantage of the invention is that it provides an intuitive interface that allows a physician to rapidly and precisely control an elongate instrument.

Another advantage of the invention is that it provides an elongate instrument that does not need to be torqued for steering, thereby allowing improved control in tortuous patient anatomy and reduced manufacturing expense.

A further advantage of the invention is that it provides a control tool that may incorporate haptic feedback to allow a physician to feel or observe contact forces applied at a distal tip of an elongate instrument.

A further advantage of the invention is that it provides an elongate instrument in which the distal tip can be shaped into a desired configuration, thereby allowing the physician to offset the distal tip from the longitudinal axis of the instrument.

A further advantage of the invention is that it provides an elongate instrument in which a physician may simultaneously control the position of the distal tip as well as its angle of attack.

Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWINGS

The drawing figures depict one or more implementations in accord with the present concepts by way of example only and not by way of limitation. In the figures, like reference numerals refer to the same or similar elements.

FIG. 1 is a schematic diagram of an exemplary control system for manipulating an elongate instrument.

FIG. 2 is an isometric view of control tool and an elongate member in a shape that corresponds to a shape of a control tool.

FIG. 3 is an isometric view of a piezoelectric actuator.

FIG. 4 illustrates the piezoelectric actuator of FIG. 3 being deflected into a bent configuration.

FIG. 5 is a cross sectional view of an actuator disposed within a portion of an elongate member.

FIG. 6 shows a perspective view of an actuator disposed within an portion of an elongate member.

FIG. 7 is an isometric view of an ionic polymer-metal composition (IPMC) film actuator.

FIG. 8 is a front view of an elongate member having a series of IPMC film actuators disposed along a portion thereof.

FIG. 9 is an isometric view of an IPMC cylindrical actuator.

FIG. 10 is an isometric view of another embodiment of an IPMC cylindrical actuator.

FIG. 11 is a front view of an elongate member having a series of IPMC cylindrical actuators disposed along a portion thereof.

FIG. 12 is a front view of a control tool.

FIG. 13 is a cross-sectional view of ball joint bending segment.

FIGS. 14 and 15 is a cross-sectional view of a cable assembly extending through a control tool shaft and bending segment respectively.

FIG. 16 is a cross-sectional view showing a transducer-actuator assembly including a cable and a pulley wheel.

FIG. 17 illustrates another embodiment of a ball joint bending segment.

FIG. 18 illustrates yet another embodiment of a bending segment including a series of ball joints.

FIG. 19 illustrates another bending segment embodiment that includes a living hinge.

FIG. 20 illustrates an elongate member colliding with a tissue structure.

FIG. 21 is a schematic diagram of a control tool having electroactive actuators disposed therein.

FIG. 22 is a schematic diagram of a control tool having ball joint actuators and an actuator-transducer cable assembly.

FIG. 23 is a front view of another bending segment including a hinge and a rotatable housing.

FIG. 24 is an isometric view of the bending segment of FIG. 23.

### DETAILED DESCRIPTION OF THE INVENTION

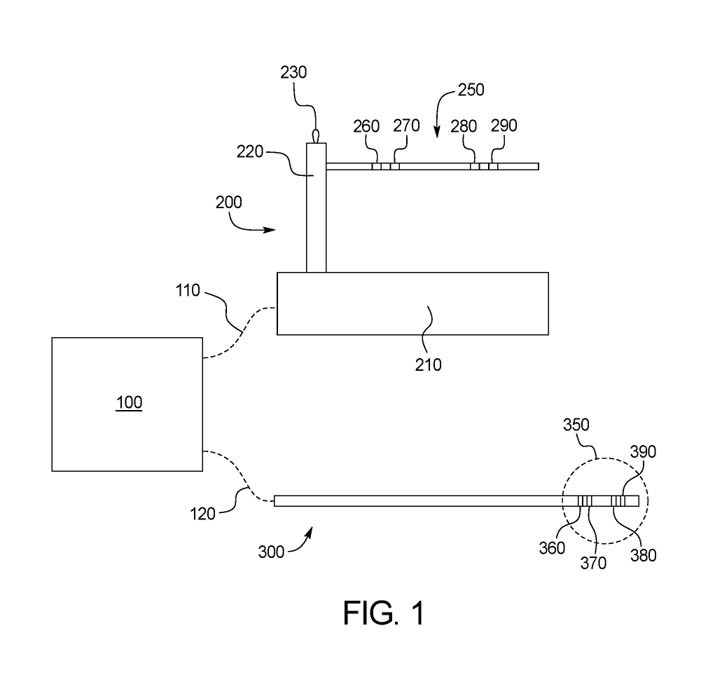

FIG. 1 shows an example of a control system including a control tool 200, a processor unit 100, and an elongate member 300. The control tool 200 may include a base 210, a housing 22, and a joystick 250. The joystick 250 may include one or more bending segments. In this embodiment, the joystick is shown having four bending segments 260, 270, 280, 290. The control tool may further include a locking switch 230. While the switch 230 is illustrated as a toggle switch, it may alternately be configured a button or any other switching means. The locking switch 230 may be used to toggle the control tool between a first configuration in which one or more of the bending segments may be freely bent and a second configuration in which they are locked in a selected position.

An elongate member 200 may be a guidewire, catheter, sheath, laparoscopic instrument, or other medical device. For purposes of illustration, the elongate member 200 is depicted as a guidewire 200, but the principles taught herein may be generalized to other devices and instruments. The elongate member 200 shown may have a distal portion 350. One or more bending segments may be positioned at the distal portion 350. In this embodiment, the distal portion is shown having four bending segments 260, 370, 380, 390. The control tool 200 may be coupled to the processor unit 100 via a wire 110, and the elongate member 300 may be coupled to the processor unit via wire 120.

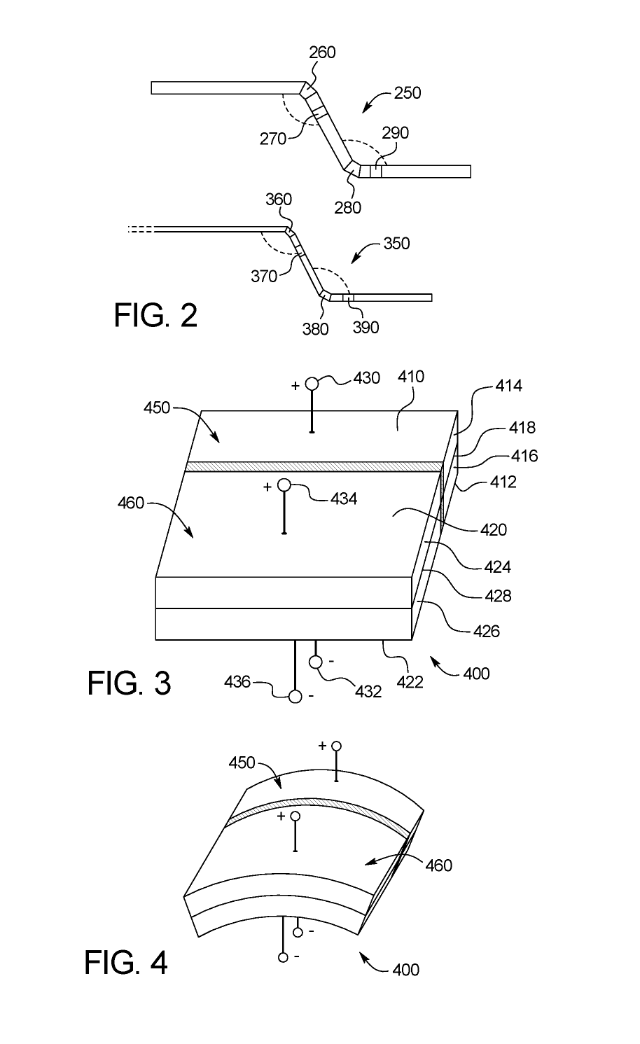

FIG. 2 shows an example of a control tool joystick 250 and an elongate member distal portion 350 in a shape that corresponds to a shape of the control tool. The joystick bending segments may include one or more transducers configured to measure the angle of bending at each joystick bending segment. The signal from each joystick transducer may be processed by the processor unit to generate an actuator signal. A given actuator signal may be calibrated to cause a respective actuator in the elongate member to bend at an angle that corresponds to the bend selected at the joystick. For example, the joystick bending segment 260 may be paired with elongate member bending segment 360, such that when a user selectively bends the joystick bending segment 260 at a given angle, the processor outputs an actuator signal to cause the elongate member bending segment 360 to bend at the same angle. This may allow the elongate member distal portion 350 to mimic or otherwise correspond to a shape selected by a user at the joystick 250.

FIGS. 3 and 4 show an example of a piezoelectric actuator unit 400. The actuator unit 400 may include an actuator 460 and a transducer 450. The actuator 460 may include a first electrode plate 420 coupled to a first wire 434, and a second electrode 422 coupled to a second wire 436. The actuator 420 may include a first piezoelectric layer 424 and a second piezoelectric layer 426. The polarities of the first and second piezoelectric layers may be opposed, so that when a voltage is applied across the first and second electrodes, one of the piezoelectric layers will tend to expand as the other tends to contract, thereby causing the actuator to bend as shown in FIG. 4. The first and second piezoelectric layers may be coupled to one another via an adhesive layer 428.

The transducer 460 may also include a first electrode coupled to a first wire 430 and a second electrode (not shown) that is coupled to a second wire 432. The transducer 460 may further include a first piezoelectric layer 414 coupled to a second piezoelectric layer 416 via an adhesive layer 418. The polarities first and second piezoelectric layers 414, 416 may be opposed such that when a bending force is applied (such as by the actuator 460), a stretching force in one of the transducer layers and a compressing force in the other transducer layer will tend to produce an output voltages of the same sign and direction. So configured, the transducer 450 may output a signal that represents the magnitude and direction in which the transducer is bent.

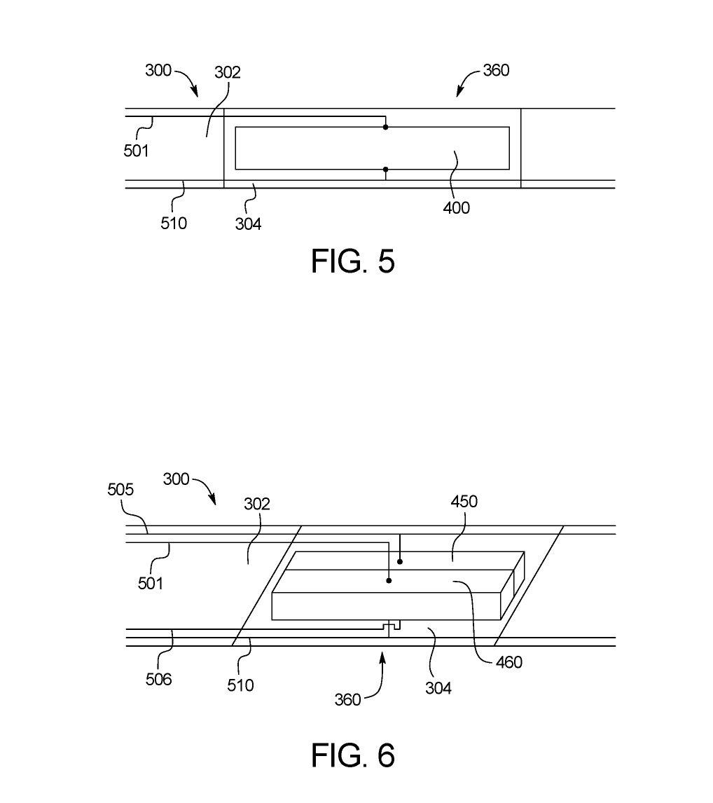

FIGS. 5 and 6 show an example of an actuator unit 400 disposed within a bending segment 360 of an elongate member 300. The elongate member 300 may comprise a body portion 302 comprised of a first material and the bending segment 360 may comprise a second, more flexible material 304. The flexible material 304 may comprise silicone rubber or other pliant material, and may be selected to maximize deflection produced by the actuator unit 400. The actuator unit 400 may be coupled to wires 501, 505, 506, 510. Wire 505 and 510 may be set to ground voltage and may pass along the length of the elongate member to couple to multiple actuator units 400 disposed at respective bending segments. Wire 501 may carry a positive or negative voltage to cause the actuator 460 to bend upward or downward respectively. Wire 506 may carry a signal produced by a transducer portion 450 to thereby measure the degree of bending produced at the bending portion.

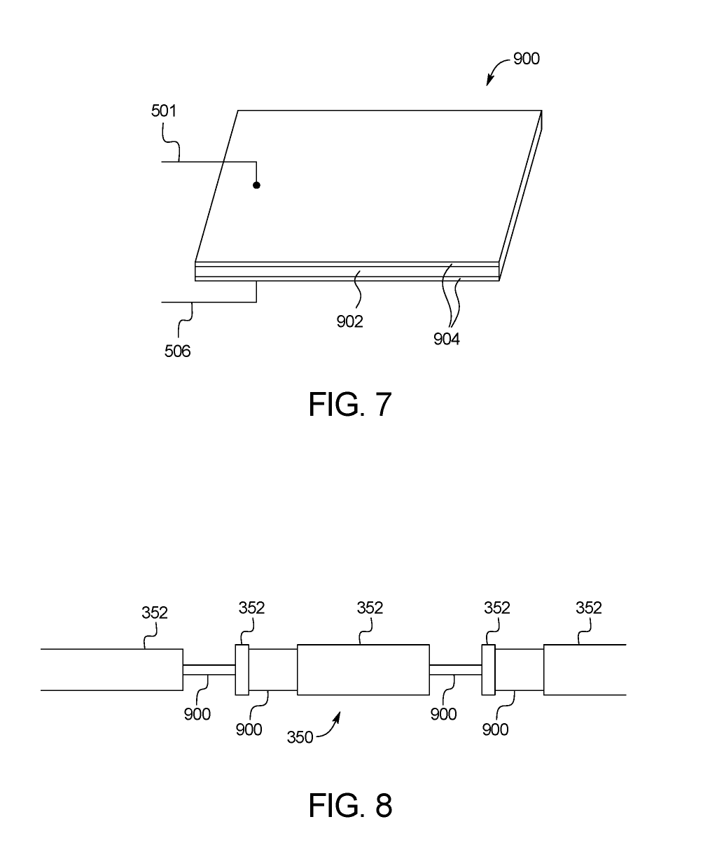

FIG. 7 shows an example of an ionic polymer-metal composition (IPMC) film actuator 900. The IPMC film actuator may comprise a polymer layer 902 surrounded by electrodes 904. Each electrode may be coupled to conductive wires 501, 506. The polymer layer 902 may comprise Nafion or other suitable polymer. In the polymer layer, positive ions naturally flow toward a selectively negatively charged electrode, dragging along solvent molecules, thereby causing the material to expand near the negative electrode and to contract near the positive electrode. This produces a natural bending motion that may be selectively controlled by controlling the charges of the two electrodes 904.

FIG. 8 shows an elongate member distal portion 350 having body segments 352 interrupted by selectively bendable EAP actuators 900. Each actuator 900 is aligned lengthwise with the longitudinal axis of the elongate member 300, and has a width greater than its depth. Adjacent actuators 900 may be disposed perpendicularly to one-another, to thereby allow the elongate member to be bent in two degrees of motion. In the exemplary embodiment of FIG. 8 wherein four bending segments are provided, the tip of the elongate member may be freely deflected.

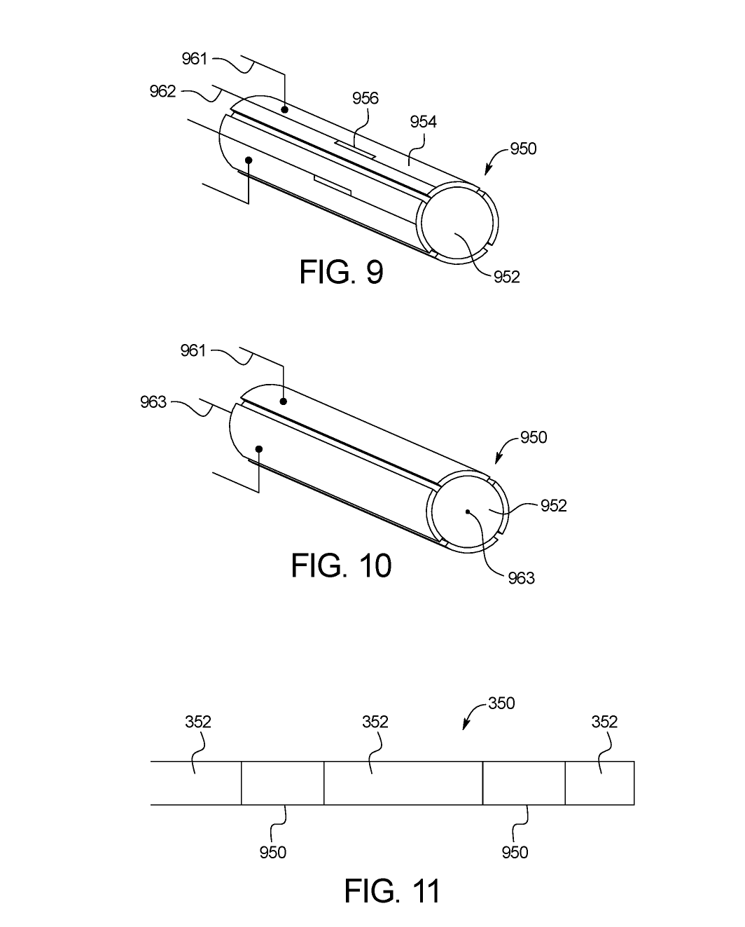

FIGS. 9 and 10 show exemplary embodiments of an IPMC cylindrical actuator 950. The actuator 950 may comprise a cylindrical polymer body 952 and one or more electrodes 954. In the exemplary embodiments shown in FIGS. 9 and 10, four electrodes 954 are provided, but other numbers of electrodes may be used. Notably, an actuator comprising at least three electrodes will allow the actuator to deflect in any direction in response to selectively applied voltages at one or more of the electrodes. Thus, the actuator 950 is configured to selectively bend along at least two degrees of motion.

In FIG. 9, the actuator 950 is illustrated having one or more gauges 956. The gauges 956 may be strain gauges or flex gauges, and may measure the degree of bending of the actuator 950. At least two gauges 956 may be provided in order to measure bending in two degrees of motion. In FIG. 10, a conductor 963 is positioned through the center of the cylindrical actuator 950. The conductor 963 measures voltage at the center of the actuator 950 relative to each of the electrodes 954 along the outer surface thereof. Because ions and solvent change position as the actuator bends, the degree of bending changes the electrical properties across the polymer body 952, thereby allowing the degree of bending to be calculated by comparing the voltage at the conductor 963 relative to the electrodes 954.

FIG. 11 shows an example of an elongate member 300 having a series of IPMC cylindrical actuators 950 disposed along a portion thereof. The actuators 950 may be disposed between elongate member body portions 352. As shown in exemplary embodiment of FIG. 11, two actuators 950 may be provided, thereby allowing free deflection at a tip of the elongate member 300. This system of bending segments allows the tip of the elongate member 300 to be freely repositioned, and further allows the angle of attack at the distal tip to be adjusted.

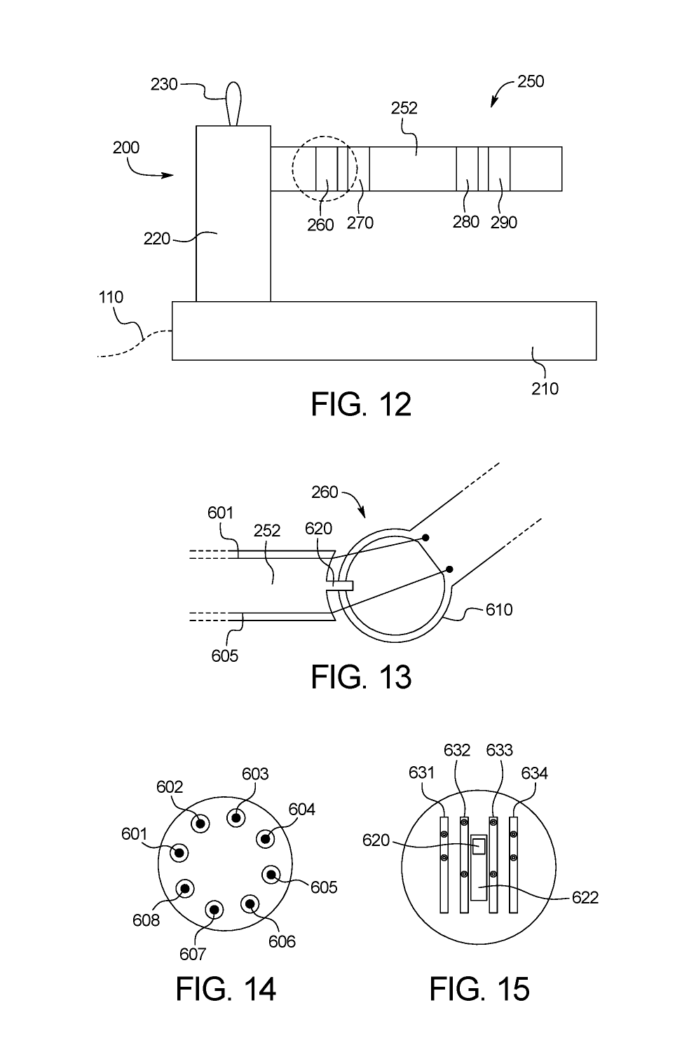

FIG. 12 shows an example of a control tool 200. As discussed with respect to FIG. 1, the control tool 200 may include a base 210, a housing 22, and a joystick 250. The joystick 250 may include one or more bending segments. In this illustrated embodiment, the joystick is shown having four bending segments 260, 270, 280, 290. Alternatively, two bending segments may be provided, wherein each of the two bending segments may be configured to bend along two degrees of motion. This configuration would be particularly advantages in combination with an elongate member comprising two IPMC cylindrical actuators 950 as described above. In the above and other embodiments, the system of joints may allow for free deflection at a tip of the joystick 250. The control tool may further include a locking switch 230. The locking switch 230 may be used to toggle the control tool between a first configuration in which one or more of the bending segments may be freely bent and a second configuration in which they are locked in a selected position.

The control tool bending segments may be manufactured according to any number of possible designs without departing from the scope of the invention. For example, FIGS. 13-15 show an example of a ball joint bending segment 260 in combination with an exemplary cable assembly 601-608. The joystick 250 may comprise shaft portions 252 through which cables 601-608 are passed. A pair of cables 601, 605 may terminate at the distal end of the bending segment 260 such that when the bending segment flexes, tension is applied to one of the paired cables while tension is relaxed on the other. In embodiments where only a single degree of motion is desired per bending segment one or more slots 631-634 may be provided in the surface of the ball joint. Additionally, a stabilizing extension 620 may be provided through a stabilizer slot 622 to restrict motion to the desired direction. The slots may extend around the majority of the ball surface, thereby allowing the joint to bend in excess of 90 degrees. Other joint structures, such as U-shaped joints may be used in place of the slotted ball joint shown in FIGS. 13 and 15.

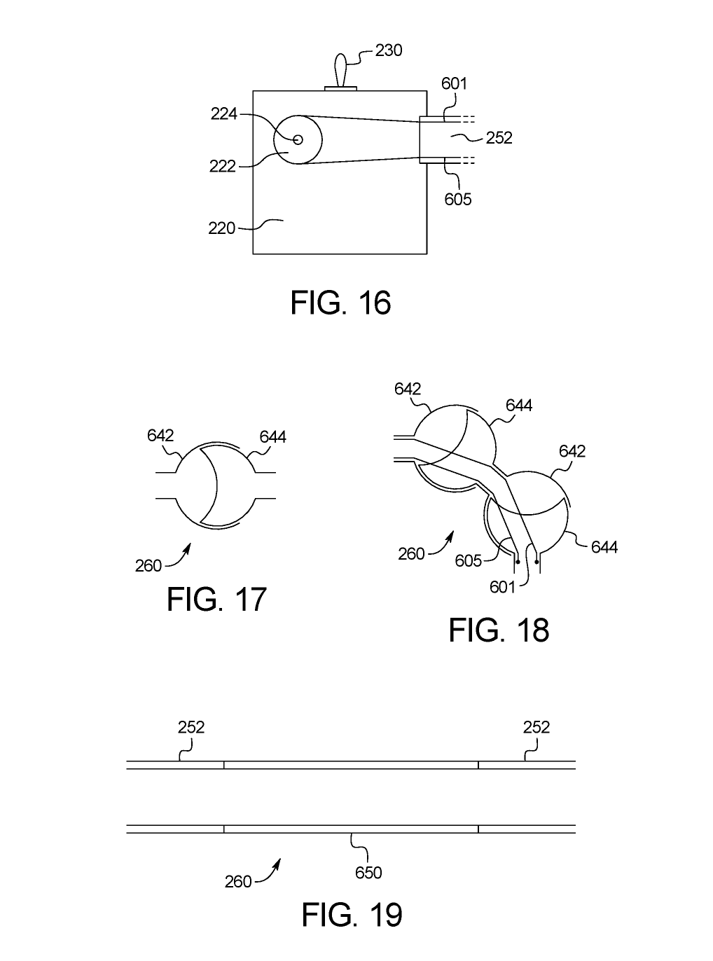

FIG. 16 shows an example of a transducer-actuator assembly including a cable pair 601, 605 and a pulley wheel 222. The paired cables 601, 605 may each be affixed to the pulley wheel, or alternatively, they may be opposed ends of a single cable. The pulley wheel 222 may be coupled to a sensor that detects its rotational position. Thus, when a bending segment is flexed, thereby applying tension to one of the paired cables 601, 605 the wheel 222 may rotate to equilibrate the tension on the paired cables, and the output of the sensor may thereby be used to calculate the degree of bending at the bending segment. Additionally, the wheel may be coupled to a motor 224, which may apply tension to one of the paired cables 601, 605 relative to the other. By applying relative tension to the cables, this force is translated to the respective bending segment to which the cable ends are affixed, thus causing the respective bending segment to flex. The locking switch 230 may be configured to hold the motor 224 at a given position, thus locking the cables, and in turn, the bending segments at a selected position. Thus, when the locking switch 230 is toggled on, the joystick may be locked in a selected shape, and when the locking switch 230 is toggled off, the joystick may again freely move in response to user inputs.

FIG. 17 shows an example of a ball joint bending segment 260 that is configured to bend in two degrees of motion. An exemplary ball joint may include a semi-spherical member 644 and a partial shell member 642. As illustrated in FIG. 18, the maximum bend for this joint design may be limited to less than 90 degrees due to contact between the shell and the joystick shaft. If a greater range of motion is desired, two or more the ball joints may be arranged in series as illustrated in FIG. 18. If multiple ball joints are used in series, the cables may be affixed at the terminus of the distal-most joint for the respective bending segment.

FIG. 19 shows an example of a bending segment comprising a living hinge 650. The shaft portions 252 of the joystick may comprise a relatively rigid material such as stainless steel or PVC. The living hinge 650 at the bending segment 260 may comprise a flexible material such as silicone rubber. Thus, a bending force applied to the bending segment 260 may naturally cause the flexible material of the living hinge 650 to flex. Gauges or cables may be used to measure the degree of flexing at the living hinge 650 as described with respect to any of the above embodiments.

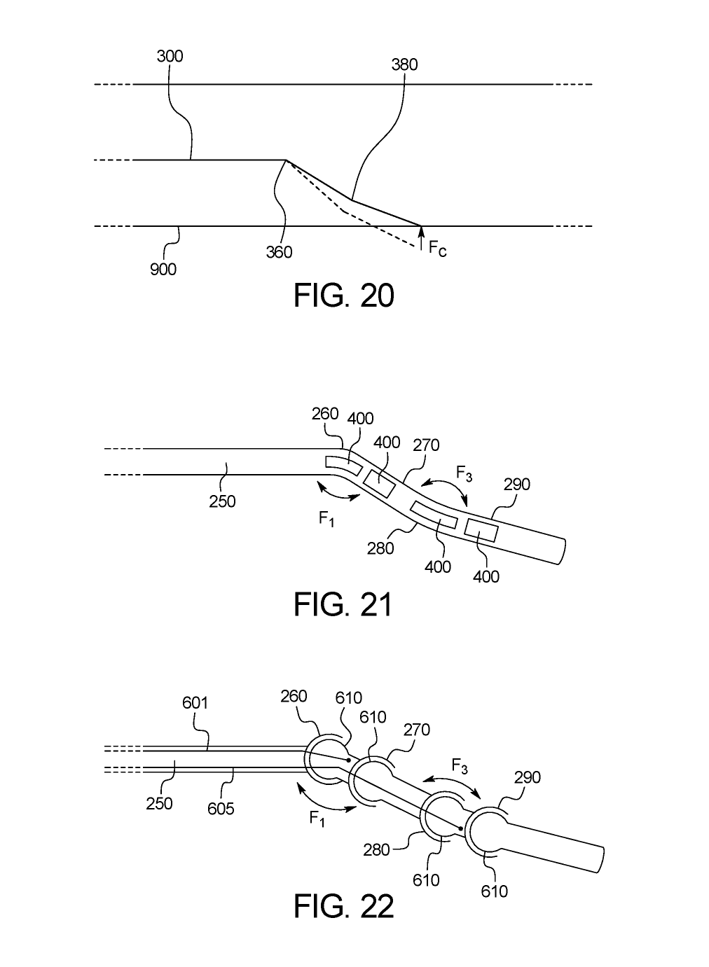

FIG. 20 shows an example of an elongate member 300 colliding with a tissue structure such as a blood vessel wall 900. When a tip of an elongate member 300 contacts with a tissue structure, the tissue structure applies a contact force F_{c}. The dotted line in FIG. 20 represents the position of elongate member 300 absent the contact force F_{c}, while the solid line represents the actual position due to the contact force F_{c}. In the illustrated example, the force F_{c} causes the angle at the bending segments 360 and 380 to be reduced. One or more transducers may be provided to measure the observed bending at each bending segment. The transducers may be strain or flex gauges, piezoelectric transducers, or other known transducers. The processor unit 100 may be programmed to calculate the contact force applied to each bending segment by comparing the observed degree of bending with the expected degree of bending based on the applied actuator signal. Upon calculating the contact force at each elongate member bending segment, the processor unit 100 may apply feedback signals to one or more actuators positioned within the control tool 200 such that the contact forces may be reproduced by actuators at the control tool 200.

FIGS. 21 and 22 show exemplary embodiments of control tools 200 that include actuators disposed therein for applying bending forces at joystick bending segments 260, 270, 280, 290. In the exemplary embodiment shown in FIG. 21, piezoelectric actuators 400 may be provided to apply force at one or more of the bending segments. In the exemplary embodiment shown in FIG. 22, meanwhile, cables 601, 605 may be provided. The force applied at each joystick bending segment may mimic or otherwise correspond to the contact force applied at each respective bending segment of the elongate member. In this manner, contact forces encountered by the elongate member may be reproduced at the joystick. Thus, the system may comprise a haptic feedback system that allows the physician to feel when the elongate member collides with tissue or other structures.

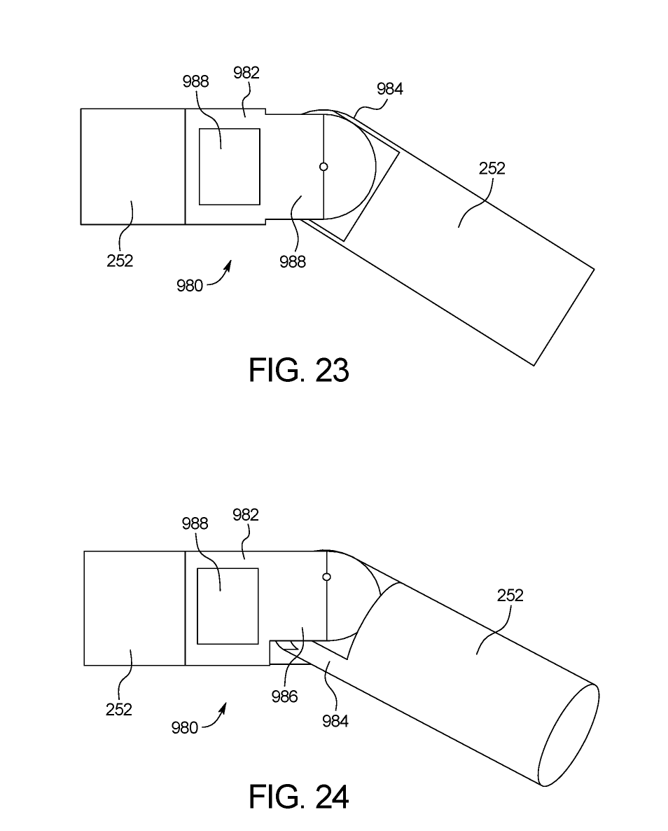

FIGS. 23 and 24 show another exemplary embodiment of a control tool bending segment including a hinge joint 984, 986 and a rotatable housing 982. The hinge joint may comprise semi-circular projection 984 positioned within a fork 986, and a pin may extend through the projection 984 and the fork 986 to pivotably couple the elements together. The shaft body portions 252 of the joystick 250 may be attached to opposing ends of the hinge joint. Additionally, a rotatable housing 982 may be rotationally coupled to a proximal or distal shaft body portion 252 to thereby allow the hinge joint to rotate the plane in which it pivots. By combining rotational movement and pivotable movement, the distal portion of the joystick shaft may be bent in any direction relative to a proximal portion of the joystick shaft. Sensors may be provided to detect the rotational position of the housing 982 and the pivotable position of the hinge joint 984, 986. In this manner, the direction and degree of bending at the joint is obtained in spherical coordinates, and this output may be translated to orthogonal coordinates at the processor unit 100 in order to determine the appropriate actuator signal to communicate to the elongate member 300.

Additionally, a motor 988 may be provided within the rotatable housing 982. The motor 988 may apply a rotational force to the housing 982 relative to the joystick body portion 252 to control the rotational position therebetween, and may further apply a torqueing force at the hinge joint 984, 986 to control the pivotable position therebetween. In this manner, the motor 988 may apply a resistance force in a given direction and magnitude in order to provide haptic feedback that simulates a contact force received at the distal tip of the elongate member 300.

It should be noted that other joint designs, including but not limited to universal joints (U-joints), may be used without departing from the scope of the invention.

In use, a physician or other user may advance an elongate member 300 through a lumen or opening in a patient's body toward an interventional site. At the interventional site, or at a location along the path of travel toward the interventional site, the user may determine that there is a need to manipulate the distal tip of the elongate member 300. The user may manipulate a control tool 200 by selectively apply bending inputs at a joystick 250 of the control tool 200. The control tool may include sensors for measuring bending inputs applied at bending segments along the joystick 250. The control tool 200 may output a bending signal to a processor unit 100. The processor unit 100 may receive the bending signal and produce an actuation signal based on the user's manipulation of the control tool. The actuation signal may be configured to cause one or more actuators in an elongate member 300 to bend in a manner that mirrors, simulates, or otherwise correlates to bending inputs received at the control tool. In this manner, the user may thereby selectively manipulate elongate member 300—and in some embodiments, the distal end thereof—to a desired position, shape, and angle of attack. The user may continue to apply bending inputs at the control tool 200 in order to selectively manipulate the elongate member 300. In this manner, a user may navigate an obstacle or perform another interventional technique.

The method may also include providing haptic feedback at the control tool 200. For example, the elongate member 300 may be advanced to contact a tissue structure, such that the tissue structure applies a contact force to the elongate member 300. The elongate member 300 may include sensors arranged at one or more joints to produce detection signals that indicate the degree of bending observed at the joints. The processor unit 100 may be configured to calculate the contact force by comparing an observed bending measurement to an expected measurement that may be determined on the basis of a selectively applied actuation signal. The processor unit 100 may generate a feedback signal based on the received detection signals, and the feedback signal may be configured to cause actuators arranged on the control tool 200 to apply bending forces that mirror, simulate, or otherwise correlate to bending inputs received from the elongate instrument 300. In this manner, a contact force applied to an elongate instrument 300 by an external structure may be simulated or reproduced in a portion of a control tool 200 that the physician grasps or otherwise observes.

It should be noted that various changes and modifications to the presently preferred embodiments described herein will be apparent to those skilled in the art. Such changes and modifications may be made without departing from the spirit and scope of the present invention and without diminishing its attendant advantages.

I claim:

1. A system for performing minimally invasive surgery, the system comprising:
a control tool comprising:
a first control tool bending segment; and
a first control tool transducer configured to generate a first control tool deflection signal based on manipulation of the first control tool bending segment;
an elongate member for insertion into a body lumen, the elongate member comprising a first elongate member bending segment at a distal portion, and a first elongate member actuator configured to apply a force at the first elongate member bending segment; and
a processor unit in communication with the control tool and the elongate member, wherein upon receipt of the deflection signal, the processor generates a first elongate member actuator signal configured to cause the first elongate member bending segment to move in accordance with the deflection signal.

2. The system of claim 1 wherein:
the control tool comprises a first control tool actuator configured to apply a force to the first control tool bending segment;
the elongate member further comprises a first elongate member sensor configured generate a first elongate member deflection signal corresponding to a deflection of the first elongate member bending segment; and
the processor unit is configured to receive the first elongate member deflection signal, and to apply a first control tool actuator signal configured to effect a deflection at the first control tool bending segment that corresponds to the deflection of the first elongate member bending segment.

3. The system of claim 2, wherein the control tool is configured to produce a tactile response when the elongate member contacts an object.

4. The system of claim 3, wherein the tactile response comprises at least one bending opposition force that corresponds to a contact force resulting from the elongate member contacting the object.

5. The system of claim 1, wherein the first elongate member actuator comprises an electroactive material.

6. The system of claim 1, wherein the control tool further comprises a second control tool bending segment, and the elongate member comprises a second elongate member bending segment.

7. The system of claim 6, wherein each of the first and second elongate member bending segments comprises an electroactive actuator having a length, a width, and a depth that is less than the width; and
the first and second actuators are disposed lengthwise along a longitudinal axis of the elongate member, and the width of the first actuator is disposed perpendicularly to the width of the second actuator.

8. The system of claim 7, further comprising third and fourth elongate member bending segments, the first, second, third, and fourth elongate member bending segments being arranged to allow free deflection at a tip of the elongate member.

9. The system of claim 1, wherein the first elongate member bending segment is configured to bend in both a first direction and a second direction perpendicular to the first in response to one or more actuator signals applied by the processor unit.

10. The system of claim 9, wherein the first elongate member bending segment comprises a substantially cylindrical ionic polymer-metal composite actuator.

11. The system of claim 10, further comprising a second elongate member bending segment, the second elongate member bending segment being configured to bend in both the first and second directions, wherein the first and second elongate member bending segments are arranged to allow free deflection at a tip of the elongate member.

12. The system of claim 2, wherein the control tool comprises a series of one or more cables, the series of cables being arranged to measure deflection at the first control bending segment and to apply a force at the first control too bending segment.

13. The system of claim 11, wherein each cable is threaded through a pulley wheel, the pulley wheel being coupled to a sensor for measuring deflection of the pulley wheel, the pulley wheel further being coupled to a motor such that the pulley wheel may be actively rotated to apply a tension force to the cable.

14. The system of claim 1, wherein the control tool has a first operating configuration in which the first bending segment may be freely bent, and a second operating configuration in which the first bending segment is locked at a selected position.

15. The system of claim 13, further comprising a switch, wherein the switch may toggled to alternate the control tool between the first operating configuration and the second operating configuration.

16. The system of claim 1, wherein the first control tool bending segment is configured to bend only within a first plane, and the control tool further comprises a second bending segment that is configured to bend only within a second plane that is perpendicular to the first plane.

17. The system of claim 15, further comprising third and fourth control tool bending segments, the first, second, third, and fourth control tool bending segments being arranged to allow free deflection at a tip of the control tool.

18. The system of claim 1, wherein the first control tool bending segment is configured to bend in both a first direction and a second direction perpendicular to the first direction.

19. The system of claim 19, wherein the control tool further comprises a second bending segment that is configured to bend in both the first and second directions, the first and second bending segments being arranged to allow free deflection at a tip of the control tool.
